(19) United States
(12) Patent Application Publication
(10) Pub. No.: US 2006/0067879 A1
(43) Pub. Date: Mar. 30, 2006

(54) Diagnosis of diseases associated with COX-2 expression

(76) Inventors: Hildegard M. Schuller, Knoxville, TN (US); George W. Kabalka, Knoxville, TN (US)

Correspondence Address: HOWARD EISENBERG, ESQ., 2206 APPLEWOOD COURT, PERKASIE, PA, 18944, US

(21) Appl. No.: 10/951,088

(22) Filed: Sep. 27, 2004

(51) Int. Cl. A61K 51/00; A61K 49/00

(52) U.S. Cl. 424/1.11; 424/9.1

(57) ABSTRACT

Derivatives of cyclooxygenase-2 inhibitor compounds are made incorporating a label that can be identified upon administration into the body of a mammal and subsequent binding of the compounds with cyclooxygenase-2 that is overexpressed due to a disease condition such as cancer.

### GOVERNMENT FUNDING

Pursuant to 35 U.S.C. §202, it is hereby acknowledged that the U.S. Government has certain rights in the invention described herein, which was supported in part by grants from the National Cancer Institute.

### FIELD OF THE INVENTION

The invention pertains to the field of diagnosis of disease. More particularly, the invention pertains to the field of diagnosis of diseases associated with expression of cyclooxygenase-2.

### BACKGROUND OF THE INVENTION

Cyclooxygenase (COX) is the enzyme that catalyzes the rate limiting step in prostaglandin synthesis, converting arachidonic acid into prostaglandin H2, which is then further metabolized to prostaglandin E2 (PGE2), PGF2α, PGD2, and other eicosanoids. COX exists in the body in two distinct isoforms, COX-1 and COX-2. COX-1 is expressed constitutively in many tissues and is responsible for basal prostaglandin production required for tissue homeostasis. COX-2 is an inducible isoform. The expression of COX-2 is stimulated by various growth factors, cytokines, and tumor promoters. COX-2 has been found to be associated with various inflammatory processes, including rheumatoid and osteoarthritis, with ischemic conditions, such as myocardial infarct and central nervous system ischemia, and with cancers, such as adenocarcinomas of the lung, pancreas, colon, prostate, and breast.

COX inhibitors, and specifically COX-2 inhibitors, have been utilized extensively as therapeutic agents to treat pain and inflammation, such as that associated with arthritis. COX-2 inhibitors have also been disclosed to be useful in the prevention and treatment of neoplasia. For example, Siebert, U.S. Pat. No. 5,972,986, discloses that COX-2 inhibitors are useful in treating or preventing neoplasias that produce a prostaglandin or express a cyclooxygenase. Such neoplasias include brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, and renal cell carcinoma.

Ristimaki, U.S. Pat. No. 6,416,961, reported that overexpression of COX-2 may be utilized as a diagnostic marker for certain gastrointestinal cancers. Ristimaki discloses a method whereby COX-2 overexpression may be detected from a patient biopsy or brush sample that is obtained during gastroscopy or gastric lavage, and that such overexpression may be utilized as an indication of the presence of gastric carcinoma. Ristimaki discloses that overexpression of COX-2 in human carcinoma appears to be restricted to the human gastrointestinal tract.

As disclosed by Ristimaki, the detection of increased expression of the COX-2 enzyme, and thus to diagnose gastrointestinal cancer, requires the removal of a tissue sample from a patient and the introduction of a catheter in order to obtain the sample. This method of detection suffers from several disadvantages. Of primary importance is that the method of Ristimaki is applicable only for cancers from which such a sample may be readily obtained. Thus, the method of Ristimaki may be used to detect cancers of the stomach but is unsuitable for detection of cancers of solid organs or of cancers of the thoracic cavity. A second disadvantage is that endoscopy is an anxiety-producing and uncomfortable procedure for a patient. Often the patient is sedated and the pharyngeal area is anesthetized. Such sedation and pharyngeal anesthesia can lead to a potentially disastrous aspiration of stomach contents into the trachea, and therefore endotracheal intubation is often performed in patients undergoing gastric endoscopy.

A significant need exists for a non-invasive method for determining the presence of an elevated COX-2 enzyme expression in a patient and for localizing such elevated expression to a particular site within the body of the patient. Such a non-invasive method could be used as a routine screening procedure to detect in an early stage the presence of diseases associated with increased expression of COX-2, such as cancer, cardiovascular and neurodegenerative diseases, and rheumatoid and osteoarthritis.

### BRIEF DESCRIPTION OF THE FIGURES

FIG. 1 shows the structure of the COX-2 inhibitor celecoxib and preferred radiohalogenated derivatives of celecoxib in accordance with the invention.

FIG. 2 shows the structure of the COX-2 inhibitor rofecoxib and preferred radiohalogenated derivatives of rofecoxib in accordance with the invention.

FIG. 3 shows the structure of the COX-2 inhibitor valdecoxib and preferred radiohalogenated derivatives of valdecoxib in accordance with the invention.

FIG. 4 is a series of 4 graphs showing the binding of a radiohalogenated derivative of celecoxib in blood, lung, liver, and pancreas in control hamsters and in hamsters pre-treated with the tobacco carcinogen 4-(methylnitrosamino)-1-(3-pyridyl)-1-butanone (NNK).

FIG. 5 shows a planar nuclear medicine image of a healthy control hamster 2 hours following a subcutaneous injection with ¹²³I-labeled celecoxib.

FIG. 6 shows a planar nuclear medicine image of a hamster that had been treated for 10 weeks with the tobacco carcinogen 4-(methylnitrosamino)-1-(3-pyridyl)-1-butanone (NNK) 2 hours following a subcutaneous injection with ¹²³I-labeled celecoxib.

### DESCRIPTION OF THE INVENTION

In one embodiment the invention is a labeled, such as a radiolabeled, chemical compound, wherein the chemical compound binds to COX-2 and is an inhibitor of COX-2.

As used in this specification, the term “COX-2 inhibitor” refers to those chemical agents that bind to the cyclooxygenase-2 enzyme and inhibit the cyclooxygenase activity of this enzyme. For purposes of this specification, a COX-2 inhibitor may have effects other than inhibition of cyclooxygenase-2. For example, a COX-2 inhibitor that is suitable for the present invention may also inhibit the COX-1 isoenzyme or may have additional pharmacologic effects unrelated to cyclooxygenase-1 or cyclooxygenase-2. Such non-specific COX-2 inhibitors are not preferred. Thus for example, indomethacin, a chemical compound that binds to both COX-1 and COX-2 receptors, is suitable although not preferred as the COX-2 inhibitor of the invention. Similarly, other chemical compounds that are non-specific COX-1 and COX-2 inhibitors, such as piroxicam, brand name FELDENE™ (Pfizer, New York, N.Y., U.S.A.) and tenoxicam, are conceived to be suitable for the present invention.

COX-2 specific inhibitors are preferred for the various embodiments of the invention. As used herein, a “COX-2 specific inhibitor” is one that demonstrates an IC50 ratio of COX-2/COX-1 of less than 2%. Examples of specific, and thus preferred, COX-2 inhibitors that are suitable for present invention include rofecoxib, brand name VIOXX™ (Merck & Co., Inc. Whitehouse Station, N.J., U.S.A.); celecoxib, brand name CELEBREX™ (Pfizer); valdecoxib, brand name BEXTRA™ (Pharmacia Corp., Peapack, N.J., U.S.A.); paracoxib, brand name DYNASTA™ (Pharmacia Corp.); etoricoxib, brand name ARCOXIA™ (Merck & Co., Inc.); NS-398 ((N-(2-cyclohexyloxy-4-nitrophenyl) methane sulphonamide); as well as other COX-2 inhibitors, either those that are presently known or those that will be discovered in the future.

Labels that are suitable for the invention include any label that can be detected on or in the body of a mammal by a non-invasive means, that is by a means that does not require removal of a tissue sample from the body. Examples of suitable labels include biotin labels, chemiluminescent labels, radioisotope labels, fluorescent labels, and nuclear magnetic resonance labels. Because radioisotope labels can readily be detected from within the body of mammal by non-invasive means, such labels are preferred.

Radioactive labels that are suitable for the method of the invention include radioactive halogens such as radioactive fluorine, iodine, and bromine, for example ¹⁸F, ⁷⁶Br, ¹²³I, and ¹²⁴I, radioactive nitrogen, for example ¹³N, radioactive oxygen, for example ¹⁵O, and radioactive carbon, for example ¹¹C. Because of their relatively long half-lives, labels of radioactive halogens are preferred.

The chemical compound may be labeled, such as radiolabeled, by any method by which the label may be adhered or connected to the chemical compound. Methods for labeling chemical compounds, such as biotin labeling, fluorescent labeling, radioisotopically labeling, chemiluminescent labeling, or nuclear magnetic resonance labeling, are well known in the art and may be readily adapted by one skilled in the art to label a COX-2 inhibitor in accordance with the invention. Commercial kits, such as those marketed by Pierce Biotechnology, Inc., Rockford, Ill., U.S., are available that provide for the labeling of chemical compounds, and such kits may be used to label a COX-2 inhibitor.

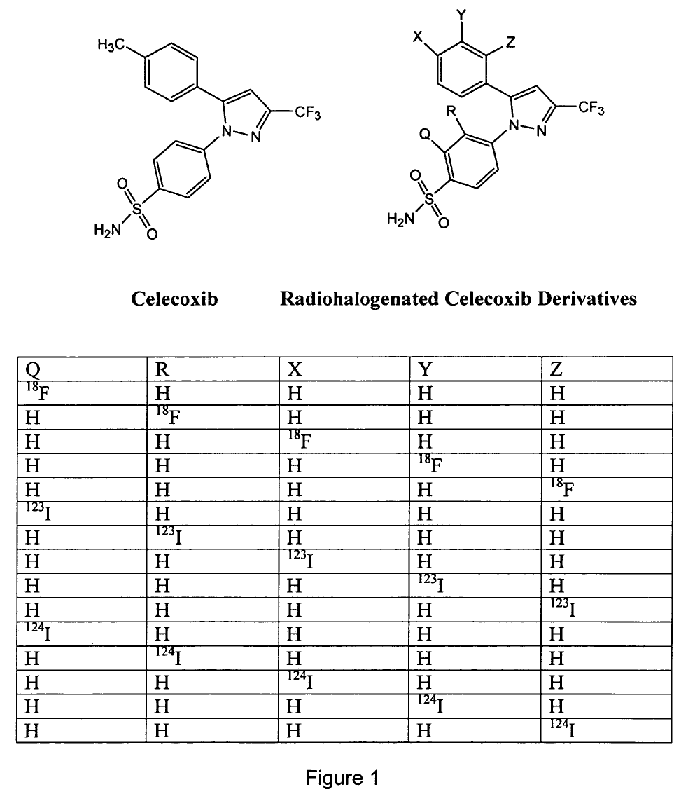

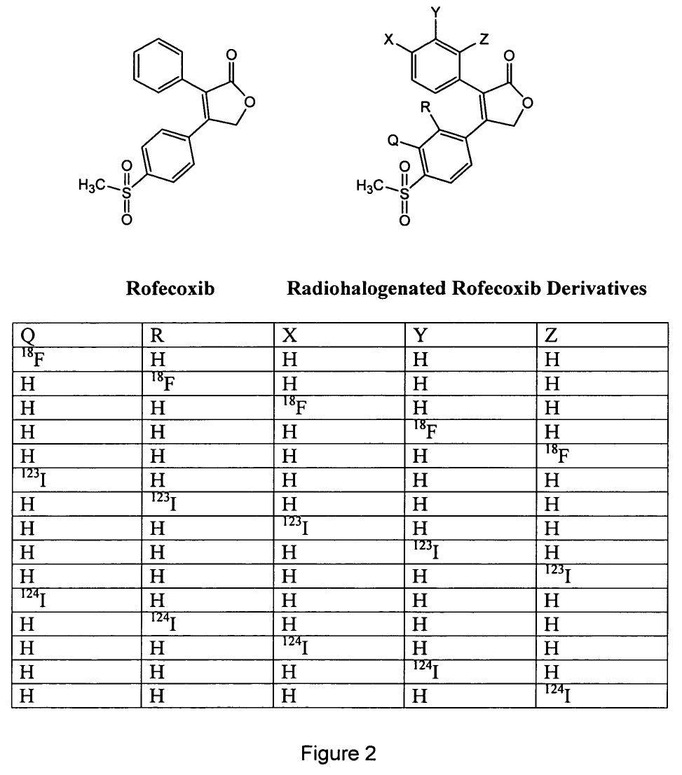

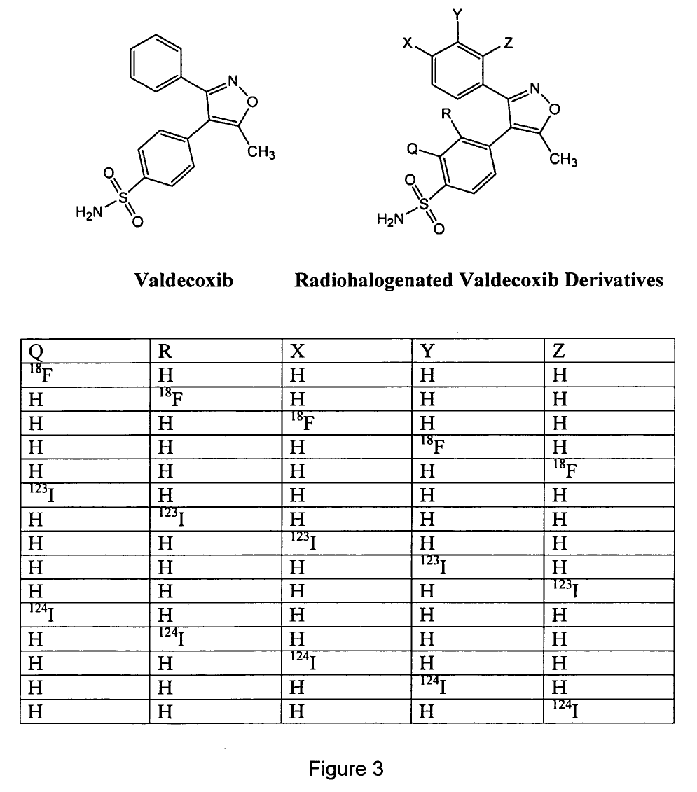

Examples of preferred radiolabeled specific COX-2 inhibitors are shown in FIG. 1, FIG. 2, and FIG. 3. FIG. 1 shows the chemical structure of celecoxib and examples of positions on celecoxib where a radiolabel may be attached to produce the radiolabeled COX -2 inhibitor of the invention. As shown in FIG. 1, the celecoxib molecule may be radiolabeled, such as with ¹⁸F, ⁷⁶Br, ¹²³I, or ¹²⁴I, in any of the positions on celecoxib designated Q, R, X, Y, or Z.

FIG. 2 shows the chemical structure of rofecoxib and examples of positions on rofecoxib where a radiolabel may be attached to produce the radiolabeled COX-2 inhibitor of the invention. As shown in FIG. 2, the rofecoxib molecule may be radiolabeled, such as with ¹⁸F, ⁷⁶Br, 123I, or ¹²⁴I, in any of the positions on rofecoxib designated Q, R, X, Y, or Z.

FIG. 3 shows the chemical structure of valdecoxib and examples of positions on valdecoxib where a radiolabel may be attached to produce the radiolabeled COX-2 inhibitor of the invention. As shown in FIG. 3, the valdecoxib molecule may be radiolabeled, such as with ¹⁸F, ⁷⁶Br, ¹²³I, or ¹²⁴I, in any of the positions on valdecoxib designated Q, R, X, Y, or Z.

In another embodiment, the invention is a method for adhering a label, such as a radioactive atom, to a COX-2 enzyme within the body of a subject. According to this embodiment of the invention, a labeled chemical compound that binds to COX-2 is administered to a subject and the labeled COX-2 inhibitor is permitted to circulate within the body of the subject and to bind to COX-2 present within the body of the subject.

In accordance with the invention, a labeled COX-2 inhibitor molecule, as described previously, is administered to a mammal and is permitted to interact with and bind to COX-2 enzyme present within the body of the mammal. In this manner, the label that is present on the COX-2 inhibitor molecule is caused to adhere, directly or indirectly, to the COX-2. The method of the invention is applicable to any mammalian subject. Preferably, the mammal is a human. Other mammals that are also suitable for the method of the invention include domesticated animals such as cattle, horses, goats, sheep, pigs, dogs, and cats.

The labeled COX-2 inhibitor molecule may be administered in any manner that will cause it to come into contact and be capable of binding to COX-2 within the body of the mammal. For example, the labeled COX-2 inhibitor may be administered intravascularly, such as intravenously. Alternatively, the labeled COX-2 inhibitor may be administered orally. It is further conceived that the labeled COX-2 inhibitor may be administered by other routes such as by inhalation or by rectal or vaginal suppository to obtain labeling of COX-2 present in the respiratory system, such as the lungs, in the lower digestive tract, such as the colon, or in the female genital tract, such as in the cervix.

Preferably, the COX-2 inhibitor molecule that is administered is a COX-2 specific inhibitor. Preferred COX-2 specific inhibitors include celecoxib, rofecoxib, valdecoxib, paracoxib, etoricoxib, and NS-398.

Preferably, the label on the COX-2 inhibitor is a radioisotope label, such as radioactive fluorine, iodine, carbon, nitrogen, or oxygen. Preferably, the radioisotope label is ¹⁸F, ⁷⁶Br, ¹²³I, or ¹²⁴I.

In another embodiment, the invention is a method for diagnosing a bodily disorder that is associated with an increased expression of COX-2. According to this embodiment of the invention, a labeled chemical compound that binds to COX-2 is administered to a subject, the labeled chemical compound is permitted to circulate within the body of the subject and to bind to COX-2 enzyme present within the body of the subject, and the presence of one or more areas within the body of the patient where the labeled chemical compound has bound to COX-2 is determined. The existence of localized concentration of binding of the labeled chemical compound to COX-2 within the body establishes the presence of localized elevated expression of COX-2 at that site.

This information, either alone or with other clinical or laboratory information, permits the diagnosis of a disorder associated with elevated COX-2 expression. The method of the invention may also be used to monitor the progression of a disease process associated with elevated COX-2 expression and to determine, for example, if the disease process is progressing or is being ameliorated over time.

In another embodiment, the invention is a method to determine the responsiveness of a diseased patient to therapy with COX-2 inhibitor compounds, either before such therapy is initiated or following such therapy. If elevated COX-2 expression is determined to be present in a patient suffering from a disease, then the patient may be selectively assigned to treatment or continued treatment with a COX-2 inhibitor compound. Conversely, if elevated COX-2 expression is not discovered, then therapy with a COX-2 inhibitor compound is likely not to be successful. In such patients, treatment of the disease with other than a COX-2 inhibitor compound may be indicated.

The chemical compound that binds to the COX-2 enzyme is preferably a COX-2 inhibitor and is most preferably a COX-2 specific inhibitor, such as celecoxib, rofecoxib, valdecoxib, paracoxib, etoricoxib, or NS-398. The label may be any label that can be attached to a chemical compound and, when such labeled chemical compound is administered to a mammalian subject, can be detected within the body of the subject by non-invasive means, that is by means other than detecting the presence of the label on a sample that has been removed from the subject. Examples of such labels include biotin labels, chemiluminescent labels, radioisotope labels, fluorescent labels, and nuclear magnetic resonance labels. Radioisotope labels are preferred.

The method by which the presence of label bound to COX-2 within the body of the patient may be determined may vary depending on several factors, including route of administration of the chemical compound, location within the body where the label is concentrated, and type of label. For example, visual inspection may be the appropriate method for determination of the presence of a fluorescent or chemiluminescent label that is bound to COX-2 within the eye, such as on the retina.

In a preferred embodiment, the presence of label bound to COX-2 is determined by an imaging modality that produces an image of a portion or of the entire body of a subject. Such imaging modalities include magnetic resonance imaging (MRI), radiography such as computerized coaxial tomography (CAT), or nuclear imaging. Nuclear imaging modalities utilize a radioactive label and the presence of the label within the body of a subject is determined by a radiation detector. Examples of suitable nuclear imaging modalities include conventional nuclear medicine, positron emission tomography (PET), and single photon emission computerized tomography (SPECT).

In a most preferred embodiment, the presence of bound label within the body is determined by a computerized nuclear medicine modality, such as PET or SPECT scan, including Micro-PET and Micro-SPECT. Utilizing a radiolabeled derivative of a COX-2 inhibitor, and preferably a COX-2 specific inhibitor, as a tracer for PET or SPECT scan, the presence of a disease condition associated with overexpression of the COX-2 enzyme within a mammalian subject may be diagnosed or monitored.

Tracers, radioactively labeled chemical compounds used for PET or SPECT scanning, that are preferred for the method of the invention include radiolabeled, preferably radiohalogenated, derivatives of COX-2 specific inhibitors such as celecoxib, rofecoxib, valdecoxib, paracoxib, etoricoxib, or NS-398. Preferred radiohalogenated labels for such tracers include radioactive fluorine, iodine, and bromine, such as ¹⁸F, ⁷⁶Br, ¹²³I, and ¹²⁴I.

This method is useful to determine whether or not a patient has a disease, such as a cancer, or to differentiate patients suffering from the disease which overexpresses COX-2 from those patients suffering from the disease which does not overexpress COX-2. Because not all patients with diseases that are associated with overexpression of COX-2, such as cancers of the lung, breast, prostate, colon, or pancreas, do indeed overexpress COX-2, and because treatment with a COX-2 inhibitor of such individuals who are not overexpressing COX-2 is typically not beneficial and long-term therapy with COX-2 inhibitors may be harmful, the method of the invention may be used to determine if a patient should be treated for the disease condition with a COX-2 inhibitor.

The invention is further illustrated in the non-limiting examples that follow. The examples describe the invention with reference to a Syrian golden hamster model of pancreatic adenocarcinoma that overexpresses COX-2 and with reference to ¹²³I-labeled celecoxib. One skilled in the art will readily comprehend that the invention is applicable to mammals other than hamsters, including humans and domesticated animals such as dogs, cats, and farm animals, to labels other than radioactive labels, to radioactive labels other than radiohalogens, to detection modalities other than nuclear medicine, to nuclear medicine modalities other than PET, and to COX-2 inhibitors other than celecoxib.

### EXAMPLE 1

### Radiolabeling a COX-2 inhibitor

### Example 1a

### Synthesis of 4,4,4-trifluoro-1-(4-iodophenyl)-butane-1,3-dione

To a solution of methyl trifluoroacetate (4.23 g, 33.0 mmol) in methyl tert-butyl ether (MTBE, 60 mL) was added NaOMe in MeOH (8.82 mL of a 25% solution, 39.0 mmol). A solution of 4-iodoacetophenone (7.38 g, 30.0 mmol) in MTBE (115 mL) was added to the mixture dropwise via an addition funnel. After stirring for 48 h at room temperature, 3N HCl (14.0 mL) was added. The organic layer was extracted into ether, washed with water and then brine, dried over anhydrous MgSO₄, filtered, and concentrated in vacuo to give a brownish solid which was recrystallized from isooctane to give 8.11 g (79%) of product.

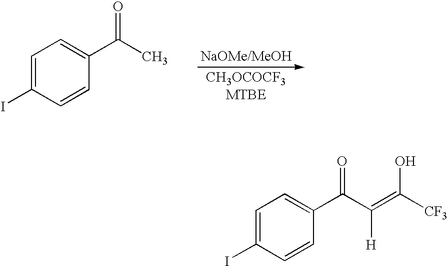

### Example 1b

### Synthesis of 4-[5-(4-iodophenyl)-3-trifluoromethyl-pyrazol-1-yl]-benzenesulfonamide

To a stirred solution of diketone (4.10 g, 12.0 mmol) in ethanol (120 mL) was added 4-(sulfamoylphenyl)hydrazine hydrochloride (2.95g, 13.2 mmol). The mixture was refluxed for 24 h. After cooling to room temperature, the reaction mixture was concentrated in vacuo. The residue was dissolved in ethyl acetate, washed with water and then brine, dried over anhydrous MgSO₄, filtered, and concentrated in vacuo to give a pale brown solid. The crude product was purified by column chromatography (30% ethyl acetate in hexane) to give 5.39 g as white solid. M.P. 180° C.

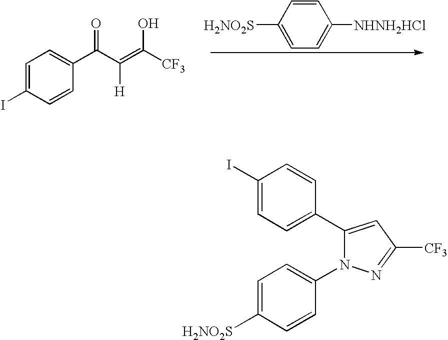

### Example 1c

### Synthesis of Celecoxib Tin Precursor

Hexamethylditin (0.98 g, 3.0 mmol) and iodocelecoxib (0.99 g, 2.0 mmol) were added sequentially to a suspension of tetrakis-triphenylphosphinepalladium (0.1 g, 0.09 mmol) in anhydrous 1,4-dioxane (15 mL). The reaction mixture was stirred at reflux under nitrogen for 0.5 h. After cooling, the mixture was filtered, the insoluble black material was washed with dioxane (20 mL), and the dioxane layers combined. Removal of dioxane solvent gave a gummy residue which was purified by chromatography(silica gel, ethyl acetate:hexane (1:1)). The solid product was recrystallized from ethyl acetate/petroleum ether. Yield (0.95 g, 90%)

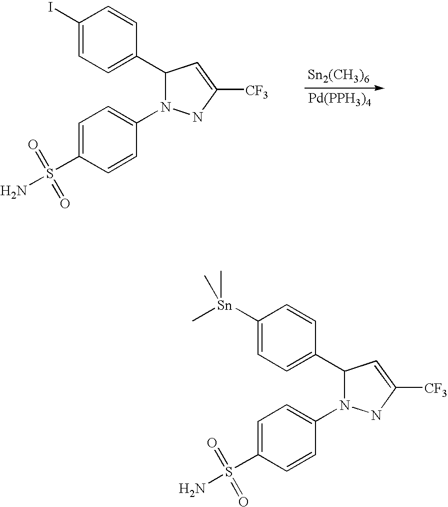

### Example 1d

### Synthesis of [¹²³I]Celecoxib

The tin precursor of Celecoxib (100 μL of 5.2×10⁻² in methanol) was placed in a 2 mL Wheaten vial containing no-carrier-added Na¹²³I (37 MBq in 0.1% aqueous NaOH). To this was added peracetic acid (100 μL, 0.3% solution in methanol). The reaction vial was sealed, covered with aluminum foil, and the mixture stirred for 5 min at room temperature. A drop of 10% aqueous sodium thiosulfate was added to decompose the excess iodine and the radioiodinated product was isolated by passing it through a silica gel Sep-pak using ethyl acetate:hexane (2:1) as eluent. The radiochemical purity of the [¹²³I]celecoxib, was determined by radio-TLC (aluminum backed silica gel plate, ethyl acetate:hexane (2:1)); R_{f}=0.62. The decay corrected radiochemical yield was determined to be 90% and radiochemical purity was >98%. The total synthesis time was 15 min.

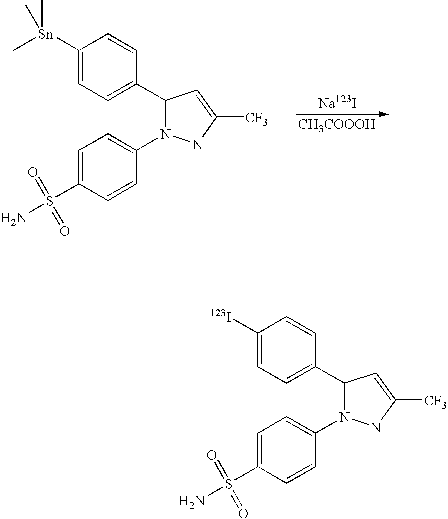

### EXAMPLE 2

### Alternate Procedure for Radiolabeling a COX-2 Inhibitor

The COX-2 inhibitor, such as celecoxib, may be radiolabeled by other methods, such as by the following series of chemical reactions.

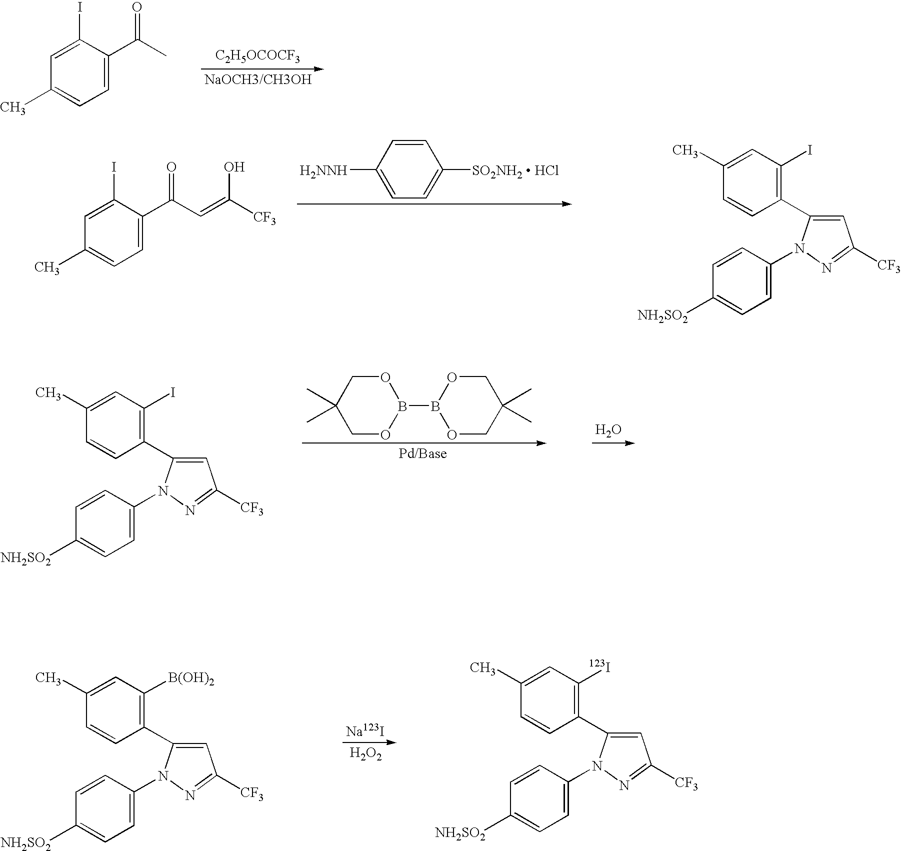

### EXAMPLE 3

### Biodistribution of Radioiodinated Celecoxib

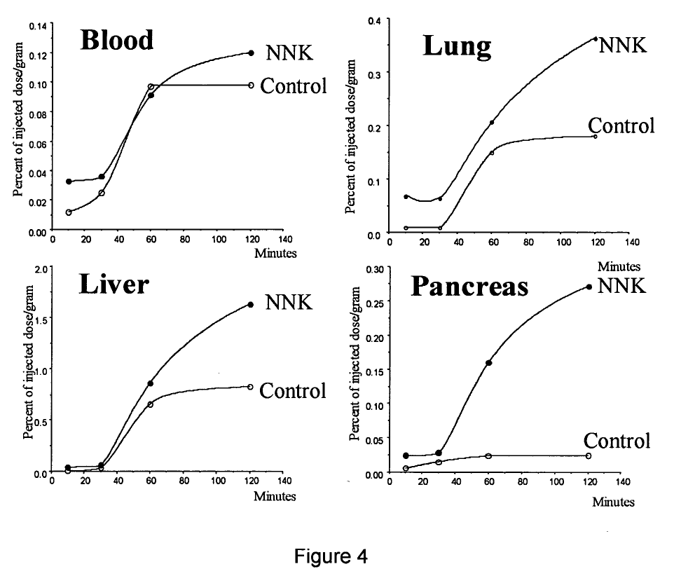

The biodistribution, over time, of radioiodinated celecoxib was compared in untreated control hamsters versus hamsters pretreated for 2 weeks with the tobacco-specific carcinogen NNK. Each of the pretreated hamsters was injected subcutaneously with 2.5 mg/100 g bodyweight NNK three times per week for two weeks. The control and the pre-treated hamsters were intravenously injected with 100 μl of a saline solution containing 5 μCi of the radioiodinated celecoxib. Three hamsters from each of the two groups were sacrificed by asphyxiation at time zero and at 30, 60, 120, and 240 minutes after administration of the radioiodinated celecoxib. Blood, lungs, liver, and pancreas were harvested and weighed. The radioactivity in each sample was quantified. The data, shown graphically in FIG. 4, indicates an increase in levels of COX-2 in the target organs of NNK-treated hamsters, in accord with previously documented ability of NNK to induce COX-2.

### EXAMPLE 4

### Induction of Animal Model of Human Cancer overexpressing COX-2

Syrian Golden Hamsters were injected subcutaneously with the tobacco-specific carcinogenic nitrosamine NNK at a dosage of 2.5 mg/100 g bodyweight three times weekly for 10 weeks. Control hamsters were untreated.

One day after the last NNK injection, the hamsters were injected intravenously with 100 liters of a saline solution containing 5 μCi of the radioiodinated ¹²³I-celecoxib tracer of Example 1. Two hours following injection of the tracer, a whole-body planar nuclear medicine image of the hamsters was obtained.

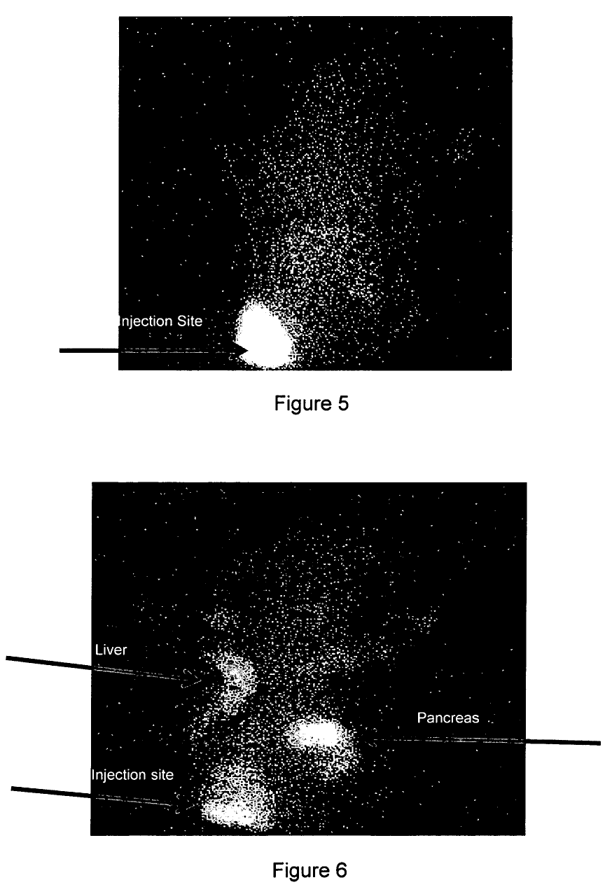

The planar nuclear medicine image of a control hamster is shown in FIG. 5 and the planar nuclear medicine image of a hamster induced to develop adenocarcinoma by injection of NNK is shown in FIG. 6. As shown in FIG. 5, healthy control hamsters showed no areas of concentration of radioiodine except at the site of injection. In contrast, as shown in FIG. 6, treated hamsters showed concentrations of radioiodine label in the pancreas and in liver. Histological and histochemical evaluation of these sites one week later (after required decay of radioactivity) confirmed the presence of early premalignant lesions (formation of pseudo-ducts) and highly positive immunoreactivity to an antibody against COX-2 in the pancreas of the NNK treated hamsters.

The diagnostic method of the invention has numerous clinical applications. It is useful for early detection of precancerous lesions even years before COX-2 overexpressing cancers develop. Thus, in high-risk individuals, the method of the invention may be used as a cancer screening method. The method of the invention is useful in patients that have been diagnosed with cancers or other conditions that are associated with overexpression of COX-2 to determine if the cancer or other condition in a specific patient is indeed overexpressing the COX-2 enzyme. Such other conditions include myocardial infarction, heart failure, rheumatoid arthritis, Alzheimer's or Parkinson's disease, and brain ischemia. If overexpression of COX-2 is present, then the patient is a likely candidate for therapy with one or more COX-2 inhibitor compounds. Alternatively, if the cancer or other condition is not found to overexpress COX-2, then long-term treatment with a COX-2 inhibitor may be contraindicated. The method of the invention is also useful to monitor patients that are receiving therapy, such as chemotherapy, for treatment of cancer or other condition associated with overexpression of COX-2.

Further modifications, uses, and applications of the invention described herein will be apparent to those skilled in the art. It is intended that such modifications be encompassed in the claims that follow.

1. A method for diagnosing a bodily disorder in a mammalian subject, which bodily disorder is associated with an increased expression of cyclooxygenase-2 (COX-2), comprising administering to the subject a labeled chemical compound that binds to the COX-2, permitting the labeled chemical compound to circulate within the body of the subject and to bind to COX-2 that is present within the body of the subject, and determining one or more areas within the body where the labeled chemical compound has bound to COX-2.

2. The method of claim 1 wherein the bodily disorder is a cardiovascular disorder.

3. The method of claim 2 wherein the cardiovascular disorder is ischemia.

4. The method of claim 1 wherein the bodily disorder is rheumatoid arthritis.

5. The method of claim 1 wherein the bodily disorder is a neurologic disorder.

6. The method of claim 5 wherein the neurologic disorder is Alzheimer's disease.

7. The method of claim 5 wherein the neurologic disorder is Parkinson's disease.

8. The method of claim 1 wherein the chemical compound is a COX-2 inhibitor.

9. The method of claim 8 wherein the COX-2 inhibitor is selected from the group consisting of rofecoxib, celecoxib, valdecoxib, paracoxib, etoricoxib, and NS-398.

10. The method of claim 8 wherein the COX-2 inhibitor is labeled with a radioactive label.

11. The method of claim 10 wherein the radioactive label is radioactive iodine, radioactive fluorine, or radioactive bromine.

12. The method of claim 11 wherein the radioactive label is iodine-123 (¹²³I) or iodine-1 24 (¹²⁴I).

13. The method of claim 11 wherein the radioactive label is fluorine-18 (¹⁸F).

14. The method of claim 11 wherein the radioactive label is bromine-76 (⁷⁶Br).

15. The method of claim 1 wherein the chemical compound is labeled with a radioactive label.

16. The method of claim 15 wherein the radioactive label is radioactive iodine, fluorine, or bromine.

17. The method of claim 16 wherein the radioactive label is iodine-123 (¹²³I) or iodine-124 (¹²⁴I).

18. The method of claim 16 wherein the radioactive label is fluorine-18 (¹⁸F).

19. The method of claim 16 wherein the radioactive label is bromine-76 (⁷⁶Br).

20. The method of claim 1 wherein the determination of the binding of the chemical compound to COX-2 is by obtaining an image of all or part of the body of the subject, wherein, within the image, an area of the body in which the chemical compound has bound to COX-2 can be distinguished from an area of the body in which there is no binding of the chemical compound to COX-2.

21. The method of claim 20 wherein the chemical compound is labeled and the determination is by obtaining an image that shows the distribution of the label throughout the body.

22. The method of claim 21 wherein the determination is by nuclear medicine imaging.

23. The method of claim 22 wherein the nuclear medicine imaging is by positron emission tomography or by single photon emission tomography.

24. A method for adhering a radioactive atom to a cyclooxygenase-2 (COX-2) enzyme molecule within the body of a subject comprising obtaining a radiolabeled chemical compound that is a COX-2 inhibitor and which COX-2 inhibitor binds to the COX-2 enzyme, administering the radiolabeled COX-2 inhibitor to a subject, and permitting the administered radiolabeled COX-2 inhibitor to adhere to COX-2 enzyme molecules within the body.

25. The method of claim 24 wherein the radiolabeled COX-2 inhibitor is administered intravascularly.

26. The method of claim 24 wherein the radiolabeled COX-2 inhibitor is administered orally.

27. The method of claim 24 wherein the COX-2 inhibitor is selected from the group consisting of rofecoxib, celecoxib, valdecoxib, paracoxib, etoricoxib, and NS-398.

28. The method of claim 24 wherein the radioactive label is radioactive iodine, radioactive fluorine, or radioactive bromine.

29. The method of claim 28 wherein the radioactive label is iodine-123 (¹²³I) or iodine-124 (¹²⁴I).

30. The method of claim 28 wherein the radioactive label is fluorine-18 (¹⁸F).

31. The method of claim 28 wherein the radioactive label is bromine-76 (⁷⁶Br).

32. A radiolabeled chemical compound which chemical compound is an inhibitor of cyclooxygenase-2 (COX-2) and wherein the chemical compound binds to the COX-2.

33. The radiolabeled chemical compound of claim 32 wherein the chemical compound selected from the group consisting of rofecoxib, celecoxib, valdecoxib, paracoxib, etoricoxib, and NS-398.

34. The radiolabeled chemical compound of claim 32 wherein the radioactive label is radioactive iodine, radioactive fluorine, or radioactive bromine.

35. The radiolabeled chemical compound of claim 34 wherein the radioactive label is iodine-123 (¹²³I) or iodine-124 (¹²⁴I).

36. The radiolabeled chemical compound of claim 34 wherein the radioactive label is fluorine-18 (¹⁸F).

37. The radiolabeled chemical compound of claim 34 wherein the radioactive label is bromine-76 (⁷⁶Br).

38. A method for determining if a patient is suffering from a bodily disorder that is associated with an increased expression of cyclooxygenase-2 (COX-2) comprising administering to the patient a labeled chemical compound that binds to the COX-2, permitting the labeled chemical compound to circulate within the body of the patient, and determining if there are present within the body of the patient one or more areas where the labeled chemical compound has bound to COX-2.
